(19) United States
(12) Patent Application Publication
(10) Pub. No.: US 2023/0211096 A1
(43) Pub. Date: Jul. 6, 2023

(54) RESPIRATORY THERAPY DEVICES

(71) Applicant: SMITHS MEDICAL INTERNATIONAL LIMITED, Ashford (GB)

(72) Inventors: Stephen James Field, Canterbbury (GB); Steven Mark Tupper, Hythe (GB); Mohammad Qassim Mohammad Khasawneh, Canterbury (GB); Usamah Hanif, Southend-on-Sea (GB)

(73) Assignee: SMITHS MEDICAL INTERNATIONAL LIMITED, Ashford (GB)

(21) Appl. No.: 18/007,551

(22) PCT Filed: Jun. 23, 2021
(86) PCT No.: PCT/GB2021/000078

§ 371 Date: Dec. 1, 2022

(30) Foreign Application Priority Data
Jul. 9, 2020 (GB) 2010540.9

(51) Int. Cl. A61M 16/00; A61M 16/08

(52) U.S. Cl. CPC A61M 16/0006; A61M 16/0866; A61M 2205/27; A61M 2205/583

(57) ABSTRACT

An oscillatory respiratory therapy device 100 has an opening 8 to atmosphere through which air passes to the user. The device has a visual indicator 20 including a paper indicator 25 and a dye reservoir 29 mounted at the opening. Before use, the opening 8 is covered by a removable cover strip 21 that prevents air flowing through the opening. The cover strip 21 is attached to the dye reservoir 29 so that the reservoir is pulled against the paper indicator 25 when the cover strip is removed, thereby the device to be used. This causes dye to flow gradually from the reservoir 29 and spread outwardly across the paper indicator 25, which is marked to indicate duration of use.

This invention relates to respiratory therapy devices of the kind having an inlet through which a user breathes, an opening to atmosphere, and a displaceable member that is displaced by breathing through the device and is arranged to produce an oscillating resistance to breathing through the device,

Positive expiratory pressure (PEP) devices, that is, devices that present a resistance to expiration through the device, are now widely used to help treat patients suffering from a range of respiratory impairments, such as chronic obstructive pulmonary disease, bronchitis, cystic fibrosis and atelectasis. More recently, devices that provide an alternating resistance to flow have been found to be particularly effective. One example of such devices is sold under the trade mark Acapella (a registered trade mark of Smiths Medical) by Smiths Medical and is described in U.S. Pat. Nos. 6,581,598, 6,776,159, 7,059,324 and 7,699,054. Other vibratory respiratory therapy devices are available, such as “Quake” manufactured by Thayer, “AeroPEP” manufactured by Monaghan, “TheraPEP” manufactured by Smiths Medical and “IPV Percussionator” manufactured by Percussionaire Corp. Alternative devices such as “CoughAssist” manufactured by Philips is also available. Respiratory therapy apparatus can instead provide an alternating resistance to flow during inhalation.

To be effective these devices must be used regularly at prescribed intervals. In the case of chronic diseases, the patient needs to use the device daily for the rest of his life in order to maintain a continuous relief. Although some of these devices can be cleaned it is important that they are replaced periodically to avoid the accumulation of contamination in or on the apparatus and to ensure that gradual wear on various parts of the device does not reach the extent where performance is affected. Typically, such devices would be replaced after six months use. The user may be instructed to make a reminder of the due date for replacement but often such reminders are lost or are not checked. This can lead to devices being used for longer that specified with the consequent possible risk of infection or inadequate therapy treatment because of wear in the therapy device. Alternatively, the device may be replaced sooner than is needed, leading to excessive cost and extra waste.

It is an object of the present invention to provide an alternative respiratory therapy device.

According to one aspect of the present invention there is provided a respiratory therapy device of the above-specified kind, characterised in that the therapy device includes a visual indicator having a part that prevents effective use of the device, and that the part is actuable both to enable use of the device and to initiate a gradual change in appearance of the visual indicator over time so as thereby to provide an indication to the user of the time from first use of the device.

The actuable part is preferably a removable cover strip. The visual indicator may include a dye reservoir and a paper indicator, the actuable part being arranged to bring the dye in the reservoir into contact with the paper indicator when the part is actuated. The dye reservoir may be arranged centrally of the paper indicator so that dye in the reservoir spreads outwardly of the paper indicator when the dye contacts the paper indicator. The dye reservoir may be positioned on one side of the paper indicator and attached with the actuable part positioned on the opposite side of the paper indicator such that, when the actuable part is pulled away from the paper indicator, it displaces the dye reservoir towards the paper indicator. The visual indicator is preferably arranged to indicate a time of up to six months from first use of the device. The actuable part is preferably located to restrict flow through the opening to atmosphere.

According to another aspect of the present invention there is provided a method of preparing a respiratory therapy device for use, the respiratory therapy device having an inlet through which the user breathes, an opening to atmosphere, a displaceable member that is displaced by breathing through the device and arranged to produce an oscillating resistance to breathing through the device, characterised in that the therapy device includes a visual indicator having a part that prevents effective use of the device, and that the method includes the step of actuating the part of the visual indicator both to enable use of the device and to initiate a gradual change in appearance of the visual indicator over time so as thereby to provide an indication to the user of the time from first use of the device.

The actuable part preferably restricts flow through the opening to atmosphere before preparation for use, and the step of actuating the part enables flow through the opening to atmosphere. The step of actuating the part preferably includes pulling away a removable cover strip.

An oscillatory PEP device and a method of preparing such a device according to the present invention will now be described, by way of example, with reference to the accompanying drawings, in which:

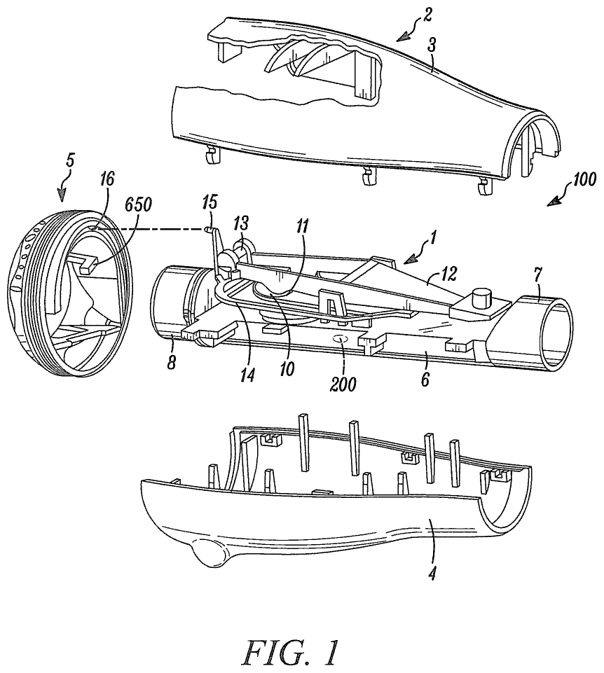

FIG. 1 is an exploded view of the device;

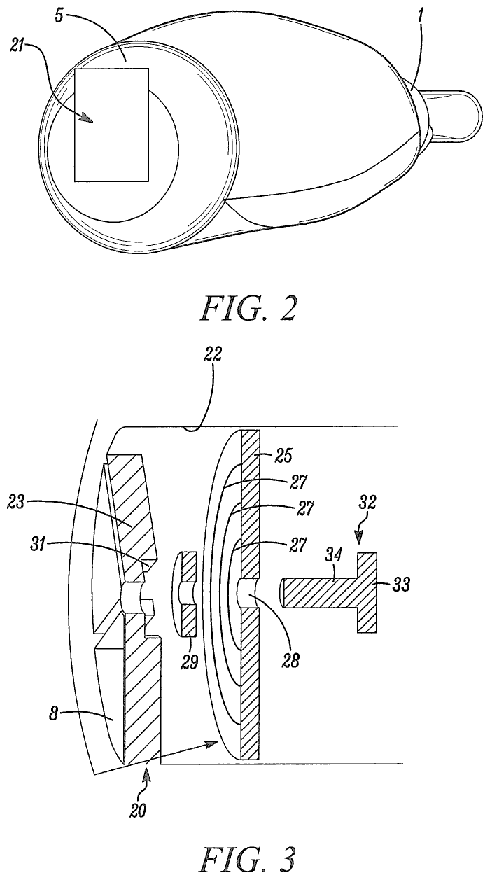

FIG. 2 is a perspective view of the device before use;

FIG. 3 is a perspective view of the machine end of the device showing the parts of the indicator in an exploded form;

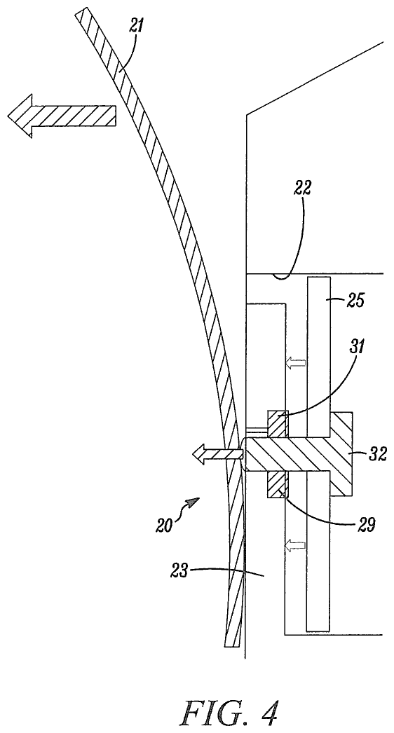

FIG. 4 is a cross-sectional side elevation view of the indicator during actuation;

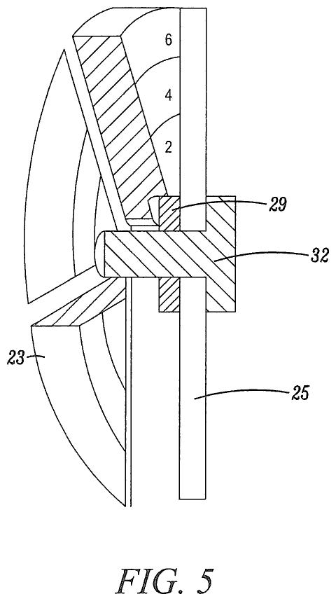

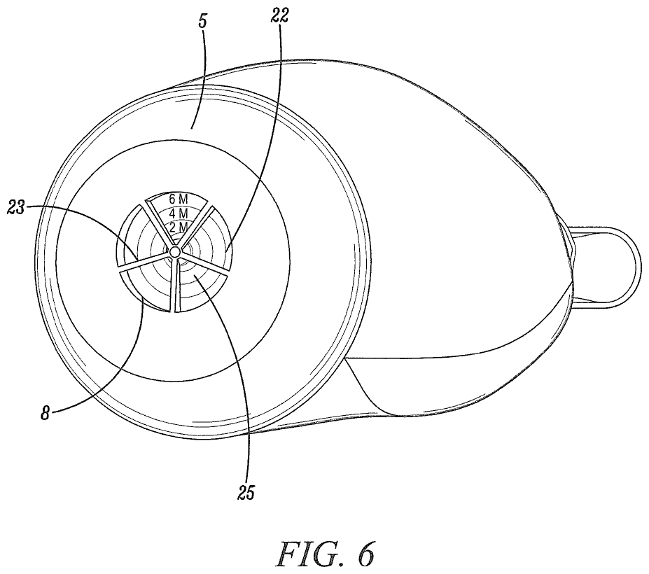

FIG. 5 is a cross-sectional side elevation view of the indicator after actuation; and

FIG. 6 is a perspective view of the machine end of the device after actuation.

With reference first to FIG. 1, the device 100 comprises a rocker assembly 1 contained within an outer housing 2 provided by an upper part 3 and a lower part 4 of substantially semi-cylindrical shape. The device is completed by an adjustable dial 5 of circular section. The rocker assembly 1 includes an air flow tube 6 with a breathing inlet 7 at one end and an inspiratory opening or inlet 8 at the opposite end including a one-way valve that allows air to flow into the air flow tube 6 but prevents air flowing out through the inspiratory inlet. The air flow tube 6 has an outlet opening 10 with a non-linear profile that is opened and closed by a conical valve element 11 mounted on a rocker arm 12 pivoted midway along its length about a transverse axis. The air flow tube 6 and housing 2 provide a structure with which the rocker arm 12 is mounted. At its far end, remote from the breathing inlet 7, the rocker arm 12 carries an iron pin 13 that interacts with the magnetic field produced by a permanent magnet (not visible) mounted on an adjustable support frame 14. The magnet arrangement is such that, when the patient is not breathing through the device, the far end of the rocker arm 12 is held down such that its valve element 11 is also held down in sealing engagement with the outlet opening 10. A cam follower projection 15 at one end of the support frame 14 locates in a cam slot 16 in the dial 5 such that, by rotating the dial, the support frame 14, with its magnet, can be moved up or down to alter the strength of the magnetic field interacting with the iron pin 13. The dial 5 enables the frequency of operation and the resistance to flow of air through the device to be adjusted for maximum therapeutic benefit to the user.

When the patient inhales through the breathing inlet 7 air is drawn through the inspiratory inlet 8 and along the air flow tube 6 to the breathing inlet. When the patient exhales, the one-way valve in the inspiratory inlet 8 closes, preventing any air flowing out along this path. Instead, the expiratory pressure is applied to the underside of the valve element 11 on the rocker arm 12 causing it to be lifted up out of the opening 10 against the magnetic attraction, thereby allowing air to flow out to atmosphere. The opening 10 has a non-linear profile, which causes the effective discharge area to increase as the far end of the rocker arm 12 lifts, thereby allowing the arm to fall back down and close the opening. As long as the user keeps applying sufficient expiratory pressure, the rocker arm 12 will rise and fall repeatedly as the opening 10 is opened and closed, causing a vibratory, alternating or oscillating resistance to expiratory breath flow through the device. Further information about the construction and operation of the device can be found in U.S. Pat. No. 6,581,598, but is not essential for an understanding of the present invention.

As so far described, the device is conventional.

With reference now also to FIGS. 2 to 6 the device also includes a visual indicator 20 that provides information of the time from first use of the device and that prevents effective use of the device until after the indicator has been actuated. The indicator 20 is mounted in or over the inspiratory inlet 8 and is arranged to block the major part of flow through the inlet until an actuable cover part 21 of the indicator has been removed. The cover part 21 need not block flow through the inlet 8 entirely but provides sufficient impediment to inhalation to indicate to the user that it needs to be removed. The cover part 21 takes the form of a flexible rectangular plastics strip accessible externally of the device and that is sufficiently stiff to be self-supporting but can be bent, gripped and pulled by the user to remove it from the device. The cover 21 extends across the opening 22 of the inlet 8 and is supported by a star-shape frame 23.

The indicator 20 also includes a circular disc 25 of absorbent indicator paper, such as a cellulose chromatography paper, and is printed on its forward surface 26 with a series of three concentric rings 27 alongside of which are printed the legends “2M”, “4M” and “6M” to indicate two months, four months and six months respectively (FIG. 6). The disc 25 is mounted just inside the opening 22 so that it is visible through the frame 23 after the cover strip 21 has been removed, or it could be mounted in other locations where it is visible externally. The disc 25 has a central aperture 28 located concentrically of the printed rings 27. The paper disc 25 may itself provide a one-way valve that flexes to permit the user to inhale through the inlet 8 around the outside of the disc but blocks flow when urged forwardly by positive pressure during expiration. Alternatively, the paper disc could be mounted on the forward surface of a flexible support such as of a resilient plastics material that provides the one-way valve at the opening 8. The indicator disc need not form a part of the one-way valve but could instead be a stiff component shaped to allow air flow around it or through it, such as through openings formed through the disc.

The indicator 20 also includes a dye or ink reservoir 29 of annular shape with a central aperture 30. The reservoir 29 is of a frangible or permeable material that releases a coloured dye within the reservoir when compressed axially, that is, along the axis of its central aperture. The dye reservoir 29 locates in a shallow, circular recess 31 formed centrally on the rear surface of the frame 23, the depth of the recess being less than the thickness of the reservoir so that the reservoir protrudes from the rear of the frame 23.

The indicator 20 is completed by a linking peg 32 with an enlarged head 33 at its forward end, which lies against the forward face of the disc 25, and a smaller diameter stem 34, which extends through the apertures 28, 30 and 24 in the disc, the dye reservoir 29 and the frame 23 respectively. The forward end of the peg 32 is attached to the rear face of the cover strip 21 (FIG. 4) by an adhesive or in some other way that enables the strip to pull the peg rearwardly when the strip is pulled but to separate from the peg when the peg reaches its limit of movement. The attachment could involve a rupturable join or a small rupturable region of the strip 21 that remains attached to the peg 32 but allows the main part of the strip to be removed.

In its normal state, as supplied and before use, the dye reservoir 29 is spaced a small distance from the forward, printed face 26 of the indicator disc 25 so the disc remains unmarked by the dye in the reservoir. In this state the cover strip 21 prevents use of the respiratory device because the strip prevents the user inhaling through the opening 8. When the device is to be used for the first time the user grips the cover strip 21 and pulls it outwardly and down away from the housing of the device as shown in FIG. 4. This pulls the peg 32 rearwardly and thereby pulls the indicator disc 25 rearwardly until it contacts the forward face of the dye reservoir 29, as shown in FIG. 5. This causes the dye in the reservoir 29 to be released from its forward face onto the centre of the rear face of the indicator disc 25. The dye and the indicator paper 25 are selected to have a very slow migration rate and, in particular, the migration rate is selected so that the dye flows outwardly to the first printed ring 27 after two months and to the outer rings after four and six months respectively. In this way, the user can readily determine the time that has elapsed since the device was first used and can replace the device when necessary, such as after six months.

This arrangement has an advantage in that it provides an indication of when the device needs to be replaced that is independent of the frequency and duration of actual use. This is important because bacteria can multiply even when the device is used infrequently.

It will be appreciated that there are other forms of visual indicator having some form of part at an opening of the respiratory therapy device to prevent effective use of the device until the part is removed or otherwise actuated and initiate change in appearance of the visual indicator so as thereby to provide an indication to the user of the time from first use of the device. Time elapse indicators of various kinds are available, for example, from Timestrip UK Ltd of Cambridge, UK.

1-10. (canceled)

11. A respiratory therapy device having an inlet through which a user breathes, an opening to atmosphere, and a displaceable member that is displaced by breathing through the device and is arranged to produce an oscillating resistance to breathing through the device, characterised in that the therapy device includes a visual indicator having a part that prevents effective use of the device, and that the part is actuable both to enable use of the device and to initiate a gradual change in appearance of the visual indicator over time so as thereby to provide an indication to the user of the time from first use of the device.

12. A respiratory therapy device according to claim 11, characterised in that the actuable part is a removable cover strip.

13. A respiratory therapy device according to claim 11, characterised in that the visual indicator (20) includes a dye reservoir and a paper indicator, and that the actuable part is arranged to bring the dye in the reservoir into contact with the paper indicator when the part is actuated.

14. A respiratory therapy device according to claim 13, characterised in that the dye reservoir is arranged centrally of the paper indicator so that dye in the reservoir spreads outwardly of the paper indicator when the dye contacts the paper indicator.

15. A respiratory therapy device according to claim 13, characterised in that dye reservoir is positioned on one side of the paper indicator and is attached with the actuable part positioned on the opposite side of the paper indicator such that, when the actuable part is pulled away from the paper indicator, it displaces the dye reservoir towards the paper indicator.

16. A respiratory therapy device according to claim 11, characterised in that the visual indicator is arranged to indicate a time of up to six months from first use of the device.

17. A respiratory therapy device according to claim 11, characterised in that the actuable part is located to restrict flow through the opening to atmosphere.

18. A method of preparing a respiratory therapy device for use, the respiratory therapy device having an inlet through which the user breathes, an opening to atmosphere, a displaceable member that is displaced by breathing through the device and is arranged to produce an oscillating resistance to breathing through the device, characterised in that the therapy device includes a visual indicator having a part that prevents effective use of the device, and that the method includes the step of actuating the part of the visual indicator both to enable use of the device and to initiate a gradual change in appearance of the visual indicator over time so as thereby to provide an indication to the user of the time from first use of the device.

19. A method according to claim 18, characterised in that the actuable part restricts flow through the opening to atmosphere before preparation for use, and that the step of actuating the part enables flow through the opening to atmosphere.

20. A method according to claim 18, characterised in that the step of actuating the part includes pulling away a removable cover strip.
